(19) United States
(12) Patent Application Publication
(10) Pub. No.: US 2011/0130697 A1
(43) Pub. Date: Jun. 2, 2011

(54) WOUND TREATMENT DEVICE AND METHOD

(76) Inventors: Richard Nagle, London (GB); Sarah Beeby, London (GB)

(21) Appl. No.: 12/746,212

(22) PCT Filed: Dec. 8, 2008
(86) PCT No.: PCT/GB2008/004049

§ 371 Date: Feb. 18, 2011

(30) Foreign Application Priority Data
Dec. 6, 2007 (GB) 0723898.3
Dec. 21, 2007 (GB) 0725063.2

(51) Int. Cl. A61N 1/36; A61F 13/00

(52) U.S. Cl. 602/53; 607/50

(57) ABSTRACT

A method of aiding the healing of a wound in a limb of a patient, the method comprises elevating the limb to a substantially horizontal or greater than horizontal angle, and applying electrical signals across the wound to stimulate healing. Also, a method and device for aiding the healing of a wound in a limb of a patient operates to generate electrical signals for a period of time at repeated intervals in accordance with a treatment regime for application of the electrical signals to electrodes applied around the wound to apply the electrical signals across the wound; to detect motion and/or elevation of the limb; and to control the application of the electrical signals to the electrodes in response to the detection to modify the treatment regime to preferentially apply the electrical signals across the wound when the limb is active and/or elevated. Further, a method and device for aiding the healing of a wound in a limb of a patient operates to generate electrical signals for a period of time at repeated intervals in accordance with a treatment regime for application to electrodes applied around the wound to apply the electrical signals across the wound; and to warn the patient before the electrical signals are going to be applied to across the wound to enable the patient to take required action in compliance with the treatment regime.

The present invention generally relates to a device and method for treating a wound on a limb, such as a leg.

It has been estimated that 600,000 individuals are affected by venous ulcers in the United States (US) alone (Falanga V, Margolis D, Alvarez O, Auletta M, Maggiacomo F, Altman M, et al. Rapid healing of venous ulcers and lack of clinical rejection with an allogenic cultured human skin equivalent. Arch Dermatol. 1998; 134:293-300). Lower extremity ulcers associated with venous insufficiency have been estimated to affect 1% to 2% of the population in developing countries and affect more than 1% of those over 60 years of age. As a result, chronic wounds of the dermal tissue present a significant public health problem that accounts for as much as 2 billion dollars in health care costs annually. Cost estimates in Europe range from approximately 1%-2% of the national health care budget (Ruckley CV. Socioeconomic impact of chronic venous insufficiency and leg ulcers. Angiology. 1997; 48: 67-69). The major costs involved in the treatment of chronic venous ulcers have been reported to result from hospitalizations, home health care and dressing changes (Olin J W, Beusterien K M, Childs M B, Seavey C, McHugh L, and Griffiths R I. Medical costs of treating venous stasis ulcers: evidence from a retrospective cohort study. Vasc Med. 1999; 4:1-7). Chronic venous ulcers arise from a number of disease states and risk factors, including diabetic neuropathy, cardiovascular ischemia, a history of deep vein thrombosis (DVT), sickle cell disease, soft tissue impairments, vascular insufficiency, and even pregnancy.

Venous ulcers are associated with a state of venous insufficiency. The venous system of the lower extremities consists of both a superficial and a deep system with perforators that connect them. Valves within the venous system direct blood from the superficial to the deep veins and back to the heart. Localised venous hypertension resulting from valvular incompetence (secondary to deep vein thrombosis or other causes) is integral to the development of tissue trauma and eventual ulceration. Normal venous circulation in the extremities is regulated by unidirectional valves that facilitate blood flow from capillary beds to the superficial system to the deep venous system, where primary valvular dysfunction occurs, localised ambulatory hypertension in the superficial veins results. Where venous insufficiency occurs, the blood flow within the deep venous system is facilitated by the pumping action of the calf muscle during physical activity and can be assisted with treatment with compression therapy and/or elevation of the leg. Prolonged periods of inactivity (e.g., extended post-operative bed rest) increase the risk of developing a thrombus in the deep veins of the calf (deep vein thrombosis or DVT). An episode of DVT can lead to the destruction of the valves within the veins, thereby allowing blood to flow in a retrograde fashion from the deep veins to the superficial system through the perforators. Instability of the valvular system further limits the ability of the calf muscle to direct blood flow adequately back to the heart. Instead, blood pools in the lower extremities, and backpressure in the capillary beds lead to edema. Fibrin is then deposited around the capillaries, limiting the transmission of oxygen and nutrients to the tissue. Pressure produced by back flow in the perforators can cause ischemia which may result in ulcerations of the overlying skin. A chronic dermatitis develops with resulting pruritus. Increased capillary proliferation, fat necrosis and fibrosis are observed at the wound edge. These changes are known as lipodermatosclerosis.

Venous ulcers are most commonly seen directly over the larger perforators above the medial malleolar location. These ulcers often fail to heal because of persistent venous hypertension and its subsequent effects (Cohen K I, Diegelmann R F, and Lindblad W J. Wound healing: biochemical and clinical aspects. WB Saunders and Company; 1992. p. 544). Venous ulcers tend to remain unhealed for many years. The complications of chronic open wounds are potentially disabling and include infections (cellulitis, sepsis, and osteomyelitis), chronic pain, impaired ability to ambulate, and in the worst case, amputation. Even in the absence of progressive disability, chronic venous ulcers tend to be resistant to treatment and frequently recur after successful therapy. It has been reported that approximately 16% of venous ulcers recur even after complete healing (Falanga et al, 1998).

Chronic wounds are complex with no single mode of action likely to ensure wound closure. Many treatment options currently available are single action in effect. The current goal of chronic wound treatments is to support the complete and sustained recovery of intact tissue structure and function. Minimizing the bacterial count of a wound, controlling pain associated with chronic wounds, proper debridement, supplying adequate blood flow to the site and understanding the underlying etiology of chronic wounds are all equally important issues in treating and promoting the healing of a wound.

Historically, the clinical approach has been aimed at reducing the risk of infection and treating secondary complications that arise from the inability of the wound to heal properly. Ideal therapy would seek to restore the impaired epithelium and promote the proliferation of epithelial cells. As standard of care, compression bandages offer a variety of therapeutic benefits to ensure adequate support of the wound environment.

There is thus an unmet medical need in subjects with unhealed venous ulcers.

One aspect of the present invention provides a method of aiding the healing of a wound in a limb of a patient, the method comprising elevating the limb to a substantially horizontal or greater than horizontal angle, and applying electrical signals across the wound to stimulate healing.

The present invention is applicable to any limb. It does however have particular applicability to the healing of wounds on the leg of a patient. Wounds in legs of patients having inferior blood circulation are particularly difficult to heal such as venous leg ulcers and diabetic foot ulcers and the present invention can assist in the healing of such wounds.

It has been recognised by the inventors that the simultaneous combination of elevation of the limb with the application of electrotherapy improves wound healing. The combined effect reduces blood pooling in the limb and stimulates both vascular and microvascular blood flow around the wound to assist in the healing process.

For the treatment of venous leg ulcers for example, the method can in one embodiment include applying a compression dressing to the limb. The compression bandage can be applied between electrotherapy treatments, although in one embodiment where the greater wound healing effect is achieved, the compression dressing is applied to the limb at the same time as the electrical signals are applied across the wound.

In one embodiment, the electrical signals are applied for a period of time at repeated intervals and the limb is elevated at said intervals. Thus repeated electrotherapy treatments can be applied over a time period.

In one embodiment, the patient is ambulatory between said intervals thus enabling the patient to carry on normal activities between treatments.

In one embodiment, the patient is provided with a warning device to warn them at the repeated intervals that the electrical signals are going to be applied to enable the patient to elevate the limb.

In one embodiment, the elevation of said limb is detected, and the electrical signals are applied when the limb is detected as elevated. This method can be employed when the patient is ambulatory and carries or wears an electrical signal generator for applying the electrical signals and avoids or reduces the occurrence of electrical signals being applied when the limb is not elevated.

Another aspect of the invention provides a method of aiding the healing of a wound in a limb of a patient, the method comprising generating electrical signals for a period of time at repeated intervals in accordance with a treatment regime for application of the electrical signals to electrodes applied around the wound to apply the electrical signals across the wound; detecting motion and/or elevation of the limb; and controlling the application of the electrical signals to the electrodes in response to the detection to modify the treatment regime to preferentially apply the electrical signals across the wound when the limb is active and/or elevated.

According to this aspect of the invention, the application of electrotherapy is controlled in dependence upon the detected motion and/or elevation of the limb. It is preferable to apply electrotherapy to the wound when the limb is active and/or elevated. The term elevated refers to the limb being placed in the horizontal position or even above, for example at up to or above 15 degrees above the horizontal position. The term active refers the activity by the muscles of the limb. This can be by way of ambulatory movement by the patient or by way of movement of only the muscles in the limb. The benefit of activity of the limb is that blood flow in the limb is increased with blood circulated through the limb via the calf pump mechanism, which has a positive effect on wound healing.

In one embodiment, the electrical signals are applied across the wound over the same average repeated intervals. This embodiment of the present invention allows the electrotherapy to be applied to the wound if the limb is not active and/or elevated between the required intervals. It is considered preferable to apply some electrotherapy even if its effect will be reduced due to the limb being static and/or lack of elevation.

In one embodiment, the patient is warned before the electrical signals are going to be applied to across the wound to enable the patient to take required action in compliance with the treatment regime. In one embodiment, the required action by the patient comprises making the limb active and/or elevated.

Another aspect of the present invention comprises a method of aiding the healing of a wound in a limb of a patient, the method comprising generating electrical signals for a period of time at repeated intervals in accordance with a treatment regime for application to electrodes applied around the wound to apply the electrical signals across the wound; and warning the patient before the electrical signals are going to be applied to across the wound to enable the patient to take required action in compliance with the treatment regime.

In one embodiment, the electrical signals are generated at repeated intervals over at least 24 hours, and the warning to the patient is suppressed over a nighttime period. This enables the patient to get an uninterrupted night sleep. In one embodiment it is not necessary to warn the patient to comply since they will inherently be complying when asleep. Further, the warning can be suppressed when it is detected that the patients limb is active and/or elevated since it is not necessary to ‘ask’ the patient to comply with the requirement to make their limb active and/or elevated.

In one embodiment, the patient is ambulatory during the treatment and hence the generator/warning device is worn or carried by the patient.

In one embodiment, the required action by the patient comprises making the limb active and/or elevated.

In one embodiment motion and/or elevation of the limb is detected and the application of the electrical signals to said electrodes is controlled in response to the detection to modify the treatment regime to preferentially apply the electrical signals across the wound when the limb is active and/or elevated.

Another aspect of the invention provides a device for aiding the healing of a wound in a limb of a patient, the device comprising an electrical generator for generating electrical signals for a period of time at repeated intervals in accordance with a treatment regime and for applying the electrical signals to electrodes applied around the wound to apply the electrical signals across the wound; and a sensor arrangement for detecting motion and/or elevation of the limb; wherein the generator is adapted to be responsive to the sensor arrangement to control the application of said electrical signals to the electrodes to modify the treatment regime to preferentially apply the electrical signals across the wound when the limb is active and/or elevated.

In one embodiment, the generator is adapted to preferentially apply the electrical signals across the wound when said limb is active and/or elevated while still applying said signals over the same average repeated intervals.

In one embodiment, a patient warning device for warning the patient before the electrical signals are going to be applied to across the wound is provided to enable the patient to take required action in compliance with the treatment regime.

In one embodiment, the required action by the patient comprises making the limb active and/or elevated.

Another aspect of the present invention provides a device for aiding the healing of a wound in a limb of a patient, the device comprising an electrical generator for generating electrical signals for a period of time at repeated intervals in accordance with a treatment regime and for applying the electrical signals to electrodes applied around the wound to apply the electrical signals across the wound; and a patient warning device for warning the patient before the electrical signals are going to be applied to across the wound to enable the patient to take required action in compliance with the treatment regime.

In one embodiment, the electrical generator is adapted to patient warning device is adapted to generate the electrical signals on multiple occasions over at least 24 hours, and the patient warning device is adapted to suppress the warning to the patient over a night time period.

In one embodiment, the device is adapted to be carried or worn by the patient.

In one embodiment, a sensor arrangement for detecting motion and/or elevation of the limb is provided; wherein the generator is adapted to be responsive to the sensor arrangement to control the application of the electrical signals to the electrodes to modify the treatment regime to preferentially apply the electrical signals across the wound when the limb is active and/or elevated.

Embodiments of the present invention will now be described with reference to the accompanying drawings in which:

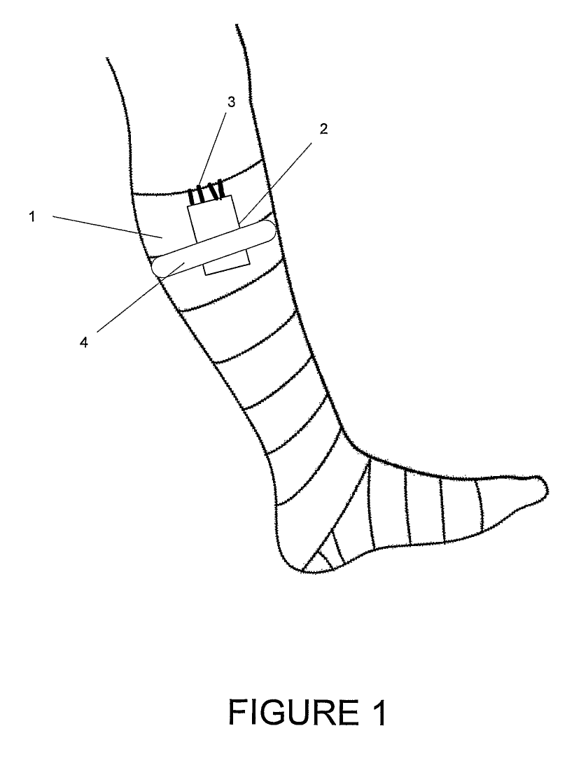

FIG. 1 illustrates an electrotherapy device used in conjunction with a compression dressing in accordance with a first embodiment of the present invention;

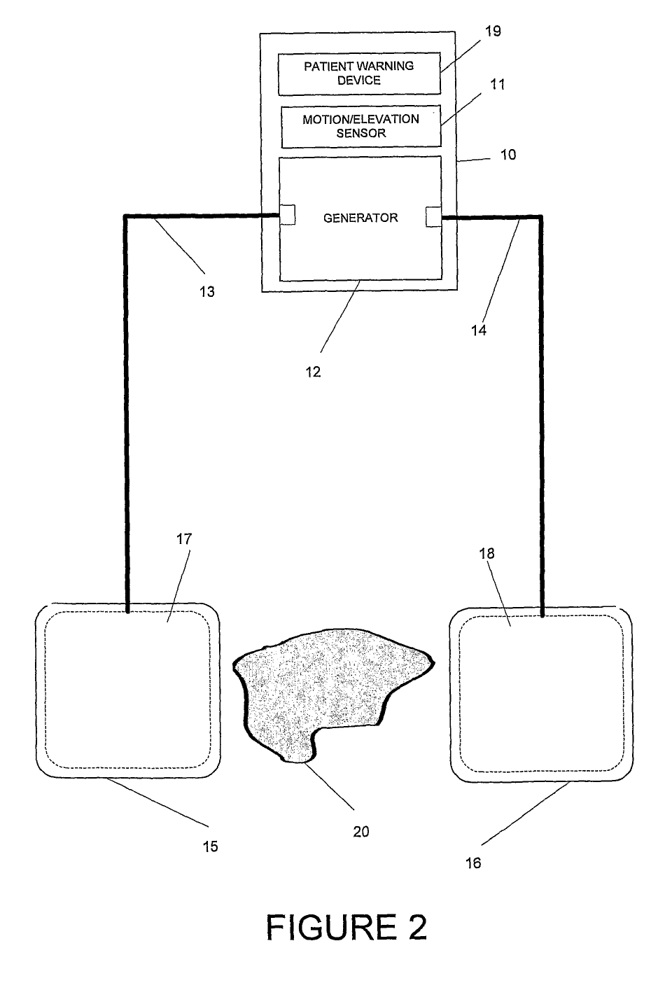

FIG. 2 is a schematic diagram of an electrotherapy device according to a second embodiment of the present invention used with electrodes positioned either side of a wound on a limb;

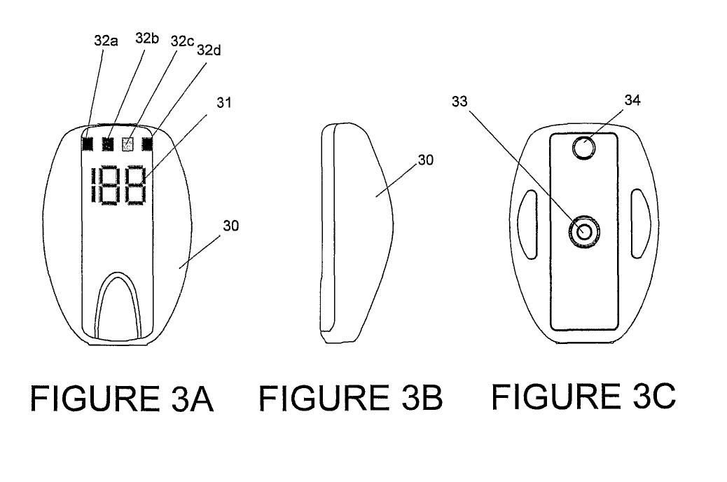

FIG. 3A to C illustrates an electrotherapy device according to a third embodiment of the invention in which, FIG. 3A is a front view, FIG. 3B is a side view, and FIG. 3C is a rear view.

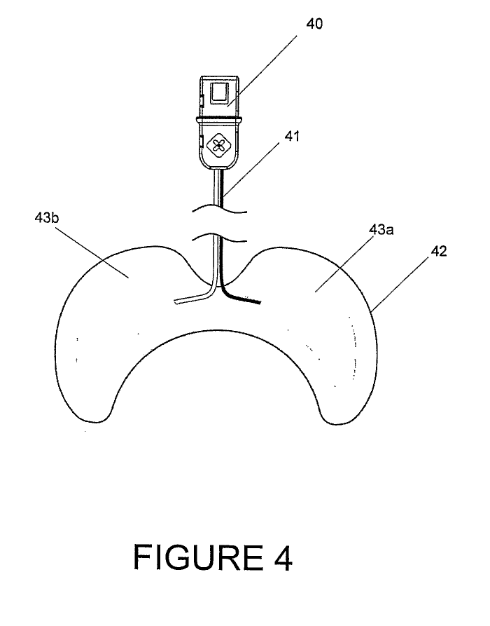

FIG. 4 illustrates the electrode for use with the third embodiment of the present invention; and

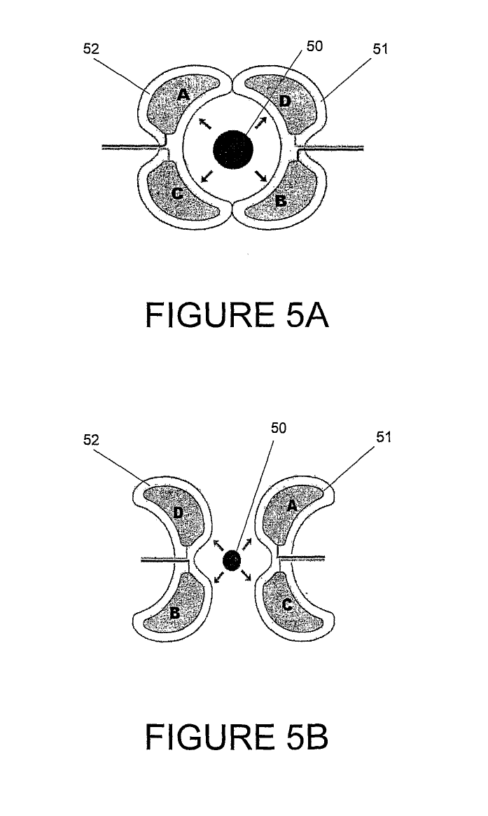

FIGS. 5A and 5B are diagrams of alternative arrangements of the electrode pair around a wound as used with the third embodiment of the present invention.

One embodiment of the present invention comprises a method of treating a wound in a limb of a patient. The present invention has particular applicability to wounds in legs caused for example by diabetic foot ulcers, venous leg ulcers or other wounds such as those caused by poor circulation. However, the present invention is applicable to other limbs such as arms.

In the method of this embodiment of the present invention, the limb of the patient is placed in an elevated position, which is preferable near horizontal or above. Electrodes are placed around a wound on a limb of the patient and an electrical generator is coupled to the electrodes so as to pass an electric current across the wound. The passage of the electric current stimulates the blood flow and accelerates the healing of the wound. By elevating the limb, pooling of blood is avoided which can act to hinder healing.

To further assist in the healing process a compression dressing such as a compression bandage or stocking is applied to the limb. This assists in returning blood from the limb. Electrotherapy can be applied alternately with the compression therapy i.e. by removing the compression dressing and applying electrodes around the wound so that an electrical current can be passed across the wound to stimulate healing and reapplying the compression dressing after the electrotherapy treatment. However, with suitably designed electrodes i.e. with electrodes that are designed to be flat and soft so that they do not cause pressure sores under a compression dressing, the compression therapy and the electrotherapy can be applied simultaneously and in combination with the elevation of the limb an improved healing environment is provided.

The electrical signals can be provided to electrodes placed either side of the wound from a static unit or a unit that can be worn or carried by the patient. The latter allows the patient to be mobile during the therapeutic process. When a compression dressing is applied the unit can be attached to the compression dressing or applied at least partially under the compression dressing. Thus in this a patient can be ambulatory between treatment periods. During a treatment period the patient will be advised to place the limb in an elevated position.

The treatment periods can be over a period of time and repeated on multiple occasions, preferably following a predetermined time interval. Between the intervals the patient can be ambulatory and can be warned e.g. by a vibratory, acoustic or visible signal (such as vibrations of the unit, an audible warning sound, or a flashing or continuous warning light on the unit) that the electrotherapy treatment is about to begin so that they can elevate the leg in preparation. The unit can also include a sensor arrangement for detecting when the limb is in the elevated position and the application of the electrical signals by the generator can be controlled in accordance with the sensed elevation so that the signals are only applied when the limb is elevated. The sensor arrangement can be used with or separately to the warning device. Thus when a patient is ambulatory during a period of time when repeated electrotherapy sessions are used, either the patient can be warned to elevate their limb or the electrotherapy session can be delayed until the patient complies with the requirement to periodically elevate their limb so that the electrotherapy sessions can take place.

The signals applied can be direct current or alternating current although alternating current has been found to have the highest therapeutic effect. It is believed that generating currents with a significant net direct current component over a long period of time should be avoided. The voltage and current used is low to reduce the likelihood of the patient feeling an unpleasant sensation. The pattern of signals applied can be designed to best suit the wound being treated.

FIG. 1 illustrates a portable electrotherapy device 2 housing an electrical signal generator used in conjunction with a compression dressing 1 in accordance with a first embodiment of the present invention. The compression dressing 1 in this embodiment comprises a compression bandage. The device 2 is attached to the top of the bandage by a retaining arrangement 4. Alternatively is the device 2 has a soft housing, it can be placed at least partially under the compression bandages. Leads 3 extend from the device 2 under the compression bandages 1 to electrodes (not shown) placed around the periphery of a wound.

FIG. 2 illustrates and electrotherapy arrangement according to a second embodiment of the present invention. A wound 20 is present on the limb of a patient. Electrodes 15 and 16 are placed either side of the wound 20. The electrodes comprise a non-conductive backing and a conductive layer 17 and 18 respectively. A unit 10 is provided housing a generator 12 for generating electrical signals for application to the electrodes 15 and 16 via leads 13 and 14. A motion and/or elevation sensor 11 is provided within the unit 10 for detecting motion and/or the angle of inclination of the unit 10. The unit 10 is designed to be worn or carried on the limb of the patient so that the unit 10 can experience the motion and/or inclination of the limb.

The generator 12 is adapted to deliver electrical signals to the electrodes 15 and 16 so that a current is applied across the wound 20. The signals can be of the form described in co-pending applications US2006/0142817 or US2007/0179585 the contents of which are hereby incorporated by reference. The generator 12 applies the signals for repeated electrotherapy periods with quiescent periods between the electrotherapy periods. For example, a patient can wear the electrotherapy apparatus for an extended period of time such as hours or days and electrotherapy can be applied at regular intervals during the period. During the extended period, the patient is encouraged to be mildly active since activity can assist in wound healing by increasing blood flow. Physical activity in patients suffering from venous leg ulcers for example is encouraged since the activity of the calf muscle during physical activity provides a pumping action to assist in blood flow in the leg. However, during the period of electrotherapy, it is preferable that the patient elevates the limb and/or keeps the limb active. The elevation of the limb assists in wound healing since it prevents pooling of blood in the limb and hence improves blood flow. Keeping the limb active assists since the muscular activity helps blood flow in the limb. Thus the generator 12 is programmed to preferentially generate the electrical signals when the motion/elevation sensor 11 detects that the limb is active and/or elevated. In one embodiment the electrotherapy signals are only applied when the motion/elevation sensor 11 detects that the limb is active and/or elevated. In an alternative embodiment, where the electrotherapy signals are applied over repeated periods of time and the treatment regime requires that the electrical signals be applied repeatedly over a period of time wherein each repeat must take place within a prescribed time interval of the last application, the detections by motion/elevation sensor 11 can be used to control the application of the electrical signals across the wound 20 if there is a period in which the limb is active and/or elevated. If not such detection of the limb being active and/or elevated occurs within the prescribed time interval, the generator is controlled to apply the electrical signals anyway since it is considered preferable to have applied some electrotherapy even if the maximum benefit may not be achieved.

A patient warning device 19 is provided in the unit 10 to enable the patient to be warned about an impending electrotherapy period to enable the patient to take appropriate action to comply with the practitioners prescribed treatment regime, such as elevate their limb and/or make the limb active during the electrotherapy session. The warning device 19 therefore monitors the intervals between electrotherapy periods so as to anticipate the generation of the electrical signals by the generator 10. The warning device 10 can take the form of any visible, audible or vibratory warning device. When the patient wears the device for an extended period of time including an overnight period, the warning device can be programmed to suppress the generation of the warning during a predetermined period overnight, which can coincide with a period in which it can be assumed that the patient is sleeping. This avoids the sleep of the patient being interrupted. Also, since the patient is likely to be lying during this period, patient compliance with the treatment regime can be assumed e.g. it can be assumed that the limb is active and/or the limb is elevated (i.e. in the horizontal position during sleep). Also, the warning device 19 can be suppressed when the motion and/or elevation sensor 11 detects that the limb is active and/or elevated since it is not necessary to ask the patient to comply with a request to make their limb active and/or elevated.

Although the second embodiment has been described with both the motion/elevation sensor 11 and the patient warning device 19, embodiments of the present invention encompass a method and a device in which only one is present.

The third embodiment of the present invention will now be described with reference to FIGS. 3 to 5 of the drawings.

The device is designed to offer medical professionals a uniform and reproducible device that enhances the effectiveness of conventional compression therapy for treatment of venous leg ulcers.

Using ambulatory electrotherapy and subject compliance monitoring features, the device brings an adjunctive therapy to compression bandages that may be used to improve healing rates.

The device is a Class IIa medical device. The Device delivers two principal treatments to the patient:

1) Electro-therapy. Defined as the delivery of micro currents (μA) to the wound area. It provides stimulation of non-healing wounds and provides analgesic effects.
2) Healing regime. The device has levels of feedback as part of the device that enables patients and clinicians/practitioners to assess adherence.

The device is an electronic module built into a case that fits onto the patient's leg under a compression bandage. The module contains three elements in one package; the Electrotherapy generator and the ‘Motion sensor’, together with Control & Display electronics. The whole is powered by a lithium primary cell.

The external appearance of the device is as per the views shown in FIGS. 3A to C. Controls are limited to one practitioner-operated switch, Switch 34 (on the face normally against the bandage and protected against inadvertent operation) which is used to obtain final practitioner information; also a separate switch, Switch 33 which is designed to detect whether the unit is properly incorporated in the bandage under compression, and is used to start operation.

Output LEDs 32a to 32d provide feedback to the Practitioner (and the Patient, in the event of a failure). LEDs 32a and 32d are red. LED 32b is green. LED 32c is yellow. The information imparted by these is described in more detail hereinafter.

On the front face adjacent to the LEDs 32a to 32d, a 7-segment digital display 31 is provided to provide patient and practitioner feedback.

The device is used in conjunction with Electrode Pads. The device uses 2 of these and each is a dual electrode. One of the pair is shown in FIG. 4. The electrode pads 42 are placed surrounding, but not touching, the wound and each comprises a pair of electrodes 43a and 43b. There is therefore the opportunity for various electric fields to be set up; in practice the two pairs of diagonally opposite electrodes will be used. Each electrode pair has two lead wires 41 terminating in a 3-pin reversible connector 40 (i.e. They can be interchanged or inserted either way up); these are plugged into the electronic module at the start of the therapy.

The diagram in FIG. 5A shows the way the different electrodes 51 and 52 are used to pass currents in different ways across the wound 50. The microampere current passes alternately from zone A to zone B, zone B to zone A; on the next session from zone C to zone D and zone D to zone C. For smaller wounds, the pads can be used back-to back to provide electrotherapy in a smaller area as shown in FIG. 5B.

An embodiment of the present invention will now be described by way of example only with reference to technical specifications of the embodiment.

### Physical Specification

| Feature | Requirement |
|---|---|
| Device Size | (H)105 mm × (W)69 mm × |
| | (D)35 mm |
| Device Weight | Approx 40 grammes |
| Pad dimensions | (H)92 mm × (W)50 mm (MAX) |

### Material Specification

| Component | Material | Comment |
|---|---|---|
| Device Casing | Injection-moulded ABS | |
| | PA-765 | |
| Device Casing | Injection-moulded PU | |
| Shroud Moulding | Injection-moulded PP | |
| Finish | General Spark texture on | |
| | all external moulded | |
| | surfaces (30 VDI) | |
| Colour | ABS parts - White | |
| | PU parts - Clear | |
| | PP (Shroud) - White | |
| Pads | Pad with two gel-based | Two Pads per Device |
| | electrodes. | |
| | Gel: AmGel 600 Series | Amgel is an exemplary |
| | Backing: | gel supplier |
| | Each pad has two lead | Lead wires are |
| | wires terminating in a 3- | PVC-coated carbon |
| | pin reversible connector | fibre (exact t.b.c) |
| | clad in a pair of Shroud | |
| | Mouldings | |
| Pad Wire length | 220 mm +10/−0 mm | |

### Product Criteria—Electrical

| Component | Specification | Comment |
|---|---|---|
| Battery | Lithium Primary Cell | Non field-replaceable |
| | CR2430 | |
| Max Voltage | 3.2 V | |
| PCB | 0.8 mm thick, comprising | |
| | surface mounted | |
| | transistors ICs, LED's, | |
| | photo devices, resistors, | |
| | capacitors | |
| Electrotherapy | Generates micro-currents | |
| generator | across the electrodes, | |
| | using specially tailored | |
| | waveforms | |
| Motion sensor | Custom opto - | |
| | mechanical sensor device | |
| | using ball bearing to | |
| | assess orientation | |
| Vibration Motor | 3 V D.C Micromotor | Supplied by LuFa (HK) |
| Opto - Display | 2½ digit, 7-segment | |
| | LED arrangement emitting | |
| | light through casework | |
| Opto - Indicators | 2 x red, 1 x yellow & 1 x | Visible from the front |
| | green LED's | face of the device |

### Product Criteria—Medical

| Component | Feature | Comment |
|---|---|---|
| Device | Fits on top of 3 layers of | Soft part of casing conforms to |
| | compression bandage, | profile of leg. Correct |
| | positioned on the top end | orientation is important |
| | of the tibia below the | |
| | knee and held in place by | |
| | the outer layer of | |
| | bandage | |
| Pads | Adhere to skin surface | Placed close to, but not on top |
| | | of, wound. |
| Biocom- | No irritation or | Conforms to EN10993-5, |
| patibility | sensitization | and -10 |

### Product Use

| | |
|---|---|
| Essential Use | Product used for treatment of venous leg ulcers (in one |
| definition | example in addition to compression bandage) |
| Controls and | Switch operated by compression (Switch 33) to activate device |
| Interaction | at start of treatment. Second switch (Switch 34) used by |
| | clinician to access data at end of 7 day treatment period. |
| Duration of Use | Device is single-use and fitted to patient for 7 days continuously. |
| Post-use procedure | At the end of the 7 day trial period, the device is removed by |
| | clinician and the post-trial data is obtained (by activating |
| | Switch 34 and reading output from 7-segment display) |

### Environmental Performance

| | |
|---|---|
| Storage and operating temps | Long Term Storage: 10° C.-32° C. |
| | Operation and Transit: 0° C.-40° C. |
| Humidity | Up to 85% non-condensing (TBC) |
| Impact resistance | Product is tested to resist direct impact on |
| | the front face similar to that incurred when |
| | accidentally striking furniture whilst |
| | wearing device |
| Drop Test | 1 m onto steel surface, each axis |
| EMC | Product is tested to EMC compliance in |
| | accordance with EN60601-1-2 |
| Recycling/re-use | The entire unit is to be viewed as |
| | disposable; however it would be desirable |
| | if the main PCBs are recyclable. |
| Water resistance | Product is designed to be ‘splash-proof’ in |
| | accordance with agreed PDD test spec. |
| | Complies with IP54 of EN 60529 |

### Therapeutic Performance

| | |
|---|---|
| Application | The unit is designed to be used in conjunction with |
| | compression bandaging of the lower leg. |
| Pain Management | With decreased pain, the patient's ability to move and adhere |
| | to a care plan is increased. This should enable improvement of |
| | the wound healing profile in conjunction with the electrical |
| | stimulation effect |
| Pressure | Correct application of compression dressing and compliance |
| | with this standard of care treatment will be measured. Existing |
| | advice applies for the application of the dressing itself |
| Electronics Module | The electronics module contains three elements in one |
| | package; the Electrotherapy generator and the ‘Motion sensor’, |
| | together with control & display electronics. |
| | The whole is powered by a lithium primary cell, which is not |
| | field-replaceable; the entire unit is to be viewed as disposable; |
| | however it would be desirable if the main PCBs are recyclable. |
| | Controls are limited to one practitioner-operated switch, |
| | Switch 34 (on the face normally against the bandage and |
| | protected against inadvertent operation) which is used to |
| | obtain final practitioner information; also a separate switch, |
| | Switch 33 which is designed to detect whether the unit is |
| | properly incorporated in the bandage under compression, and |
| | is used to start operation. Operation is started by continuous |
| | closure of Switch 33. |
| Electrode Pads (2 off) | Each of these is a dual electrode, featuring conductive gel pads |
| | with an insulating foam backing. |
| | The crescent electrodes are placed surrounding, but not |
| | touching, the wound. There is therefore the opportunity for |
| | various electric fields to be set up; in practice the two pairs of |
| | diagonally opposite electrodes will be used. |
| | Each electrode has two lead wires terminating in a 3-pin |
| | reversible connector (i.e. they can be interchanged or inserted |
| | either way up); these are plugged into the electronic module at |
| | the start of the therapy. They are designed to be a one-time fit |
| | so they cannot be removed once inserted. For this reason, they |
| | should only be plugged in once the device and pads are |
| | correctly positioned. |
| Electrotherapy | The electrotherapy generator generates micro-currents across |
| | the electrode pads, using specially tailored waveforms (see |
| | Electrotherapy treatment programs). |
| | Each electrotherapy treatment session uses one pair of |
| | electrodes only, but consecutive treatments use alternate pairs. |
| | The generator is equipped to sense an unduly high resistance |
| | between the electrodes, and to notify the patient urgently of an |
| | issue with the wound treatment. |
| Motion Sensor | The sensor is designed to detect one of four conditions of the |
| | leg: Activity, Static-Dependent, Horizontal, or Elevated. |
| | Elevated is defined as a module orientation of 15° (±2°) above |
| | the horizontal, with a hysteresis such that a return to |
| | ‘horizontal’ occurs at 10° (±2°). |
| | The results are processed and used to provide motivation to the |
| | patient, to control the electrotherapy process, and to inform the |
| | practitioner (see “Processing of the Motion sensor” below). |
| Processing of the | Every 7.5 minutes the preceding 15 samples from the sensor |
| Motion Sensor | are considered and a determination made as to the predominant |
| | condition in that 7.5 minutes. |
| | This is then used in various ways (NB electric stimulation and |
| | static dependency logging are inhibited for the first hour run): |
| | A count of the number of consecutive occurrences of ‘Static |
| | Dependent’ is updated based on each 7.5 minute interval. |
| | The count is reset if ‘Activity’, ‘Horizontal’ or ‘Elevated’ is |
| | found to be the predominate condition in a 7.5 minute slot. If |
| | the count reaches 4 (equivalent to 30 minutes) the count is |
| | reset to 0 and the vibration alarm is triggered to vibrate 3 times |
| | for 500 mS over 2.5 S. If the Static Dependent condition |
| | continues, the vibration will be repeated every 30 minutes. |
| | A total count of the number of 7.5 minute slots is kept, for use |
| | in the final practitioner report. |
| | A total count of the number of 7.5 minute slots predominately |
| | ‘Activity’ is kept, for use in the final practitioner report. |
| | A total count of the number of 7.5 minute slots predominately |
| | ‘Horizontal’ is kept, for use in the final practitioner report. |
| | A total count of the number of 7.5 minute slots predominately |
| | ‘Elevated’ is kept, for use in the final practitioner report. |
| | A timer runs, resetting every 6 hours. For the first 3 hours 5 |
| | minutes of each 6 hours, electrotherapy is inhibited from |
| | starting. After this time electrotherapy is triggered if the |
| | predominant condition in the previous 7.5 minutes is not |
| | ‘Static-dependent’. At the end of the 6 hours electrotherapy is |
| | triggered anyway. However, one and only one electrotherapy |
| | session can be started in any 6-hour cycle. |
| Patient feedback | Discrete patient feedback is provided by a vibration alarm. |
| | This is capable of reminding the patient of the need to take |
| | some action. |
| | Additional patient feedback is provided using the red centre 7- |
| | segment digital display an upside-down (i.e. patient-oriented) |
| | letter ‘F’ to indicate a terminal error has occurred, and |
| | practitioner support should be urgently requested. This |
| | indication flashes for 0.5 second every 5 seconds for 24 hours |
| | (subject to battery power availability). |
| Practitioner feedback | Practitioner feedback is triggered by closing the Switch 34 |
| | contacts on the back of the unit, and is provided using a 2½ |
| | digit (i.e. 0-199) of red 7-segment LED display, which will |
| | flash together to indicate each item of information as described |
| | in “Post Treatment Feedback” section below) |
| | After the first hour run, use of the switch terminates the |
| | monitoring and electrotherapy activities, but the unit remains |
| | capable of responding to the switch by repeating the report. |
| Processing of the | The monitoring and electrotherapy functions are started by |
| ‘Compression Sensing’ | sustained application of compression. |
| switch (Switch 33) | 1 - Within the first hour of Compression switch (Switch 33) |
| | closure, no data is logged nor electrotherapy started and |
| | release of the switch causes the unit to return to the condition |
| | as originally supplied. |
| | During this period only, the start of an ‘Elevated’ condition is |
| | indicated by an inverted ‘EL’ on the display, flashing for 0.5 |
| | second every 5 seconds for 1 minute. This is primarily to give |
| | feedback to the practitioner that the device is operational and |
| | that the patient is properly elevating. During this time the |
| | practitioner switch (Switch 34) is inactive. |
| | 2 - After the first hour, data is logged and release of Switch |
| | 33 for less than 5 minutes is ignored; for more than 5 minutes, |
| | logging and electrotherapy terminates and results in a |
| | ‘Compression fail’ indication. |
| | If a practitioner report is started within 1 hour of the end of the |
| | 5 minute period, the ‘Termination status’ (see “Post Treatment |
| | Feedback” section below) will be ‘Pass’; otherwise it will be |
| | ‘Compression Fail’. |
| Test feedback | During the state before the unit has committed to run (i.e. |
| | Switch 33 has never been held closed for at least an hour) |
| | closing Switch 33 with switch 34 pressed causes the unit to |
| | display a message and vibrate the vibrator. |
| | This is intended to give feedback that the unit is functioning |
| | and may be used at final inspection or if required at any time |
| | before switch 33 has been closed for an hour continuously. |
| | The display message will reflect the code version of the |
| | motion processor, starting with ‘100’. All the four separate |
| | LEDs will illuminate. This message is flashed as usual; the |
| | unit then returns to the normal startup condition (as if only |
| | Switch 33 was pressed). |
| Electrotherapy failure | An unsuccessful session is one where the contact-checking |
| and feedback | feature fails, indicating poor electrode-skin contact or a fault, |
| | and will cause logging and electrotherapy functions to |
| | terminate immediately, with the unit displaying the terminal |
| | error indication using the red centre 7-segment digit to display |
| | an upside-down (i.e. patient-oriented) letter ‘F’ to indicate a |
| | terminal error has occurred, and practitioner support should be |
| | urgently requested. |
| | This indication flashes every 5 seconds for 24 hours (subject to |
| | battery power availability). |
| Post-treatment | At the end of the week the practitioner removes the assembly. |
| Feedback | The practitioner switch (Switch 34) is than pressed, and the |
| | unit enters readout mode. In this mode Switch 34 is inactive; |
| | the mode is exited only by no depression of the compression |
| | switch (Switch 33) for 5 minutes. When the Switch 33 is then |
| | pressed and released the first of the practitioner report |
| | messages is displayed on the 7-segment displays and separate |
| | LEDs on the upper part of the unit for approximately 1 second. |
| | Each time the Switch 33 is pressed, the next message in the |
| | sequence flashes. |
| | When the sequence is complete, further presses of Switch 33 |
| | will cause the readout sequence to repeat. If readout is |
| | interrupted for more than 5 minutes, the unit resets so that a |
| | Switch 34 press starts the sequence anew. All other displays |
| | cease during this operation. The practitioner report uses the 7- |
| | segment displays, flashing a number (0-199) or other |
| | indication for approximately 1 second repeated for six values. |
| | These outputs are in sequence as follows: |
| | 1. Termination reason code (no upper LEDs flash): |
| | P: (pass) normal termination |
| | CF: compression fail termination |
| | EF: electrotherapy fail termination |
| | 2. ‘Total’ hours (0-199) (all four LEDs flash) |
| | 3. ‘Static-dependency’ hours (0-199) (RED upper RH LED |
| | flash) |
| | 4. ‘Horizontal’ hours (0-199) (YELLOW upper LED flash) |
| | 5. ‘Elevated’ hours (0-199) (GREEN upper LED flash) |
| | 6. ‘Activity’ hours (0-199) (RED (2) upper LH LED flash) |
| | In the cases of a count of hours, if a value exceeds 199 |
| | (unlikely), it is displayed as ‘Or’. |
| | Numeric values are expressed with leading-zero suppression |
| | and in this mode are read ‘right way up’ when the unit is held |
| | upright with sockets at the bottom |
| | The scores described above in 2-6 above are derived from the |
| | counts of 7.5 minute periods spent predominately in each |
| | condition. In each case, the number of 7.5 minutes periods is |
| | divided by 8 and rounded down to give the hours totals. As a |
| | result the total may not be exactly the sum of the sub-totals. |

### Electrotherapy Treatment

| Electrotherapy treatment | |
|---|---|
| programmes | |
| GLOSSARY | amplitude - in this context, the peak value of the current |
| | waveform |
| | commutated - reversed in sign or polarity |
| | duration - length of time a treatment phase is sustained |
| | frequency - the number of complete cycles of a waveform |
| | each second, expressed in Hz (1 Hz = 1 cycle/second) |
| | tolerance - the variation, expressed as a percentage of the |
| | nominal value, that a property of the output may acceptably |
| | deviate from that nominal, as a result of the combined effects |
| | of manufacturing variations and the effects of temperature, |
| | battery condition, etc. within the intended usage limits of the |
| | product. |
| | unipolar square wave - a waveform where a period spent at |
| | the designated amplitude alternates with the same period at |
| | an amplitude of zero; the whole repeating at the designated |
| | frequency times per second. |
| Each Treatment | Each treatment consists of three phases, each applying a |
| | unipolar square wave current, commutated every 10 S to |
| | avoid the presence of an average direct-current component. |
| | The voltage limit on the output is variable (depending on |
| | battery condition and current demand) but will not exceed |
| | 17 V nor be less than 12 V. |
| | Nominally, the three phases are as follows: |
| | Frequency 1 Hz (i.e. 500 mS of current, 500 mS of none), |
| | amplitude 100 μA, duration 300 S (5 minutes) |
| | Frequency 3 Hz (i.e. 166 mS of current, 166 mS of none), |
| | amplitude 40 μA, duration 900 S (35 minutes) |
| | Frequency 100 Hz (i.e. 5 mS of current, 5 mS of none), |
| | amplitude 320 μA, duration 2100 S (25 minutes) |
| | Each treatment therefore lasts 65 minutes. |
| | At the end of the treatment a continuity test through the |
| | electrodes is carried out, and the result used to report the |
| | electrotherapy as ‘successful’ or ‘unsuccessful’. |
| Daily Regime | Four treatments as above are carried out every 24 hours, |
| | optimally when the leg is elevated (see Section on Motion |
| | Sensor processing for the algorithm employed). Each |
| | treatment uses the alternate pair of pads to its predecessor. |
| Treatment accuracy | The accuracy of times and frequencies mentioned in the |
| | above description is to be maintained with a tolerance of |
| | ±2% throughout normal working conditions. |
| | The accuracy of currents mentioned in the above description |
| | is to be maintained with a tolerance of ±20% throughout |
| | normal working conditions. |

### Electrotherapy Development

| | |
|---|---|
| Software | The firmware for the various PIC micro-controllers is written |
| Design | in assembler using the MPLAB-IDE from Microchip. This |
| Method- | is a well-established product. |
| ology, | The firmware is modularised, to enhance clarity and re- |
| Tools and | usability. |
| Control | The firmware design avoids the use of interrupts, using |
| | instead a polling architecture which simplifies the task of |
| | code verification by reducing the incidence of asynchronous |
| | events. |

In the third embodiment, the LEDs 32a to 32D, or the digital display 31 can be used to give the patient a warning that the electrotherapy is about to begin so that the patient can move their leg into an elevated position and/or make their leg more active. Alternatively the warning vibratory device used to warn the patient to become more active can be used in a second manner to warn the patient that the electrotherapy is about to begin. The vibratory warnings can be two distinct type of vibration.

Although the present invention has been described with reference to specific embodiments, it will be understood by a skilled person in the art that modifications lie within the spirit and scope of the accompanying claims.

1. A method of aiding the healing of a wound in a limb of a patient, the method comprising:
elevating the limb to a substantially horizontal or greater than horizontal angle; and
applying electrical signals across the wound to stimulate healing.

2. A method according to claim 1, including applying a compression dressing to the limb.

3. A method according to claim 2, wherein said compression dressing is applied to the limb so as to apply compression to the limb at the same time as the electrical signals are applied across the wound.

4. A method according to claim 1, wherein said electrical signals are applied for a period of time at repeated intervals and said limb is elevated at said intervals.

5. A method according to claim 4, wherein the patient is ambulatory between said intervals.

6. A method according to claim 5, including providing the patient with a warning device to warn them at said repeated intervals that said electrical signals are going to be applied to enable said patient to elevate said limb.

7. A method according to claim 1, including detecting the elevation of said limb, and applying said electrical signals when said limb is detected as elevated.

8. A method of aiding the healing of a wound in a limb of a patient, the method comprising:
generating electrical signals for a period of time at repeated intervals in accordance with a treatment regime for application of said electrical signals to electrodes applied around the wound to apply said electrical signals across the wound;
detecting motion and/or elevation of said limb; and
controlling the application of said electrical signals to said electrodes in response to the detection to modify said treatment regime to preferentially apply said electrical signals across the wound when said limb is active and/or elevated.

9. A method according to claim 8, wherein said electrical signals are applied across the wound over the same average repeated intervals.

10. A method according to claim 8, including warning the patient before the electrical signals are going to be applied to across the wound to enable the patient to take required action in compliance with the treatment regime.

11. A method according to claim 10, wherein the required action by the patient comprises making the limb active and/or elevated.

12. A method of aiding the healing of a wound in a limb of a patient, the method comprising:
generating electrical signals for a period of time at repeated intervals in accordance with a treatment regime for application to electrodes applied around the wound to apply said electrical signals across the wound; and
warning the patient before the electrical signals are going to be applied to across the wound to enable the patient to take required action in compliance with the treatment regime.

13. A method according to claim 12, wherein said electrical signals are generated at repeated intervals over at least 24 hours, and said warning to the patient is suppressed over a night time period.

14-16. (canceled)

17. A device for aiding the healing of a wound in a limb of a patient, the device comprising:
an electrical generator for generating electrical signals for a period of time at repeated intervals in accordance with a treatment regime and for applying said electrical signals to electrodes applied around the wound to apply said electrical signals across the wound; and
a sensor arrangement for detecting motion and/or elevation of the limb;
wherein said generator is adapted to be responsive to said sensor arrangement to control the application of said electrical signals to said electrodes to modify said treatment regime to preferentially apply said electrical signals across the wound when said limb is active and/or elevated.

18. A device according to claim 17, wherein said generator is adapted to preferentially apply said electrical signals across the wound when said limb is active and/or elevated while still applying said signals over the same average repeated intervals.

19. A device according to claim 17, including a patient warning device for warning the patient before the electrical signals are going to be applied to across the wound to enable the patient to take required action in compliance with the treatment regime.

20. A device according to claim 19, wherein the required action by the patient comprises making the limb active and/or elevated.

21. A device for aiding the healing of a wound in a limb of a patient, the device comprising:
an electrical generator for generating electrical signals for a period of time at repeated intervals in accordance with a treatment regime and for applying said electrical signals to electrodes applied around the wound to apply said electrical signals across the wound; and
a patient warning device for warning the patient before the electrical signals are going to be applied to across the wound to enable the patient to take required action in compliance with the treatment regime.

22. A device according to claim 21, wherein said electrical generator is adapted to patient warning device is adapted to generate said electrical signals at repeated intervals over at least 24 hours, and said patient warning device is adapted to suppress the warning to the patient over a night time period.

23. A device according to claim 21, wherein said device is adapted to be carried or worn by the patient.

24. (canceled)
